(19) United States
(12) Patent Application Publication
(10) Pub. No.: US 2012/0046716 A1
(43) Pub. Date: Feb. 23, 2012

(54) Phototherapy Apparatus

(76) Inventor: Gordon Rex Paterson Dougal, Durham (GB)

(21) Appl. No.: 13/264,138

(22) PCT Filed: Apr. 12, 2010
(86) PCT No.: PCT/EP2010/054762

§ 371 Date: Oct. 12, 2011

(30) Foreign Application Priority Data
Apr. 14, 2009 (GB) 0906224.1
Oct. 21, 2009 (GB) 0918452.4

(51) Int. Cl. A61N 5/06; B23P 11/00

(52) U.S. Cl. 607/91; 29/428

(57) ABSTRACT

The present invention provides a customised transcranial phototherapy device which follows the contours of the patients head closely, the device comprising a ring assembly and light emitting modules positioned between an upper and lower ring of the ring assembly. The invention also includes methods of making the customised device and methods of treating various neurological conditions with the device.

The present invention relates to a phototherapy apparatus for delivering light for therapeutic purposes, and in particular to a customised or bespoke apparatus to be worn on or about the head, for the transcranial and/or transdermal delivery of therapeutic light to a site on or in an individual's head and in particular to deliver therapeutic light to selected areas of the brain. The invention includes inter alia methods of treating neurological disorders, diseases and conditions.

### BACKGROUND

Targeting tissues with phototherapy can be difficult due to the individual variation in anatomy, in addition there are often referred symptoms which identify the location of the pathology to a skilled individual but are obscure to a lay person. The “one size fits all” type of applicator does not take into consideration the lack of anatomical knowledge of the general population and “treating where it hurts” does not, in many cases address the source of the disability.

It is known in the prior art to treat the underlying brain with infrared light and to use a strapped on head-band type device or to use a helmet/hat with a chin strap or the like to secure it about an individual's head. It is also known from the prior art that 1072 nm light has been demonstrated to be therapeutic in the treatment of dementia however the delivery of the light to the target parts of the brain has hitherto proven to be difficult given the large variation in head size and cranial anatomy. Adjustable “head gear” has not proved to be sufficiently versatile or to effectively deliver therapeutic light to the target area.

Various intracranial pathologies have differing areas of neuronal atrophy and pathology, necessitating irradiation of different parts of the brain. MRI and SPECT scans are able to identify specific areas of the brain which are not functioning appropriately. Cognitively impaired individuals have problems with spatial awareness making the use of mechanical headgear even if adjustable, poorly therapeutic. Patients with neurodegenerative conditions often have poor coordination and a visible tremor, making the use of adjustable headgear almost impossible without the help of another individual.

A device which could deliver therapeutic light more efficiently and more effectively to specific and selected areas of the brain would offer improvements over the prior art.

A device which could be fitted more accurately to a particular individual and that is simple to operate, thereby allowing an individual to operate it themselves, would offer improvements over the prior art.

### BRIEF SUMMARY OF THE DISCLOSURE

In the broadest aspect of the invention there is provided a customised transcranial phototherapy device which follows the contours of the patient's head or skull closely.

According to a first aspect of the invention there is provided a phototherapy device comprising a first substantially circular ring and a second substantially circular ring that is of a smaller diameter than the first ring, each ring being sized and shaped so as to be approximately commensurate with the circumference of an individual's head and in use is able to fit over the patient's head, each ring being provided with a plurality of engaging means for receiving and securing a plurality of light emitting modules therebetween.

Reference herein to head is intended to mean the part of the head which includes the cranium enclosing the brain and the back of the skull.

Preferably, the first and second rings are constructed of a rigid or semi-rigid material selected from the group comprising silicone, resin, plastics and rubber. The material is selected to ensure comfort for the user whilst providing a certain amount of flexibility so that the device can be placed onto a user's head at the same time providing sufficient anchorage for the light emitting modules.

It will be appreciated that each ring will have an inner surface that is the surface in contact with a part of the light emitting modules and an outer surface which is, in the case of the first ring the surface most remote from the crown of an individual's head and in the case of the second ring the outer surface is the surface in closest proximity to the crown of the individual's head. Preferably the outer surfaces of both the first and second rings are substantially flat. Preferably the inner surfaces of the first and second rings are profiled according to the type of engagement means employed in the device that secure the light emitting modules between the two rings.

The first and second rings are spaced apart defining a region therebetween into which light emitting modules are placed and secured.

In one embodiment of the invention the engaging means of the first and second rings that engage with the light emitting modules are in the form of complimentary interlocking male and female projections and receiving units. Preferably the light emitting module is provided with at least one or more cylindrical projections that are received into complementarily shaped receiving sections or grooves or slots provided on an inner surface of the first and second rings.

In an alternative embodiment of the invention the engaging means of the first and second rings that engage with the light emitting modules are in the form of a rod and spindle arrangement. The light emitting modules being provided with a hollow projection on a top and bottom surface of its casing into which a rod maybe inserted, the first and second rings being provided with means for securing the rod.

Preferably, the light emitting modules may be releasably engaged with the first and second rings so that they may be replaced with ease. In some instances it is preferable to further secure the light emitting modules with an appropriate adhesive material to ensure that they are not dislodges in use.

The power supply to operate the device of the present invention may be main electricity or may be provided by a battery.

Preferably the light emitting modules comprises a housing or casing within which there is provided a plurality of light emitting means selected from the group comprising LEDs, lasers, laser diodes, light emitting polymers, light emitting organic or non-organic polymers and nanocrystals. More preferably the light emitting means are LEDs.

Preferably, the light emitting means emit light of between 1020 nm to 1120 nm and more preferably light centred around a peak wavelength of 1072 nm.

Preferably, the light source can be pulsed or continuous wave.

Preferably, the light emitting module further includes a fan or cooling element.

Preferably, the phototherapy device further includes any one or more of the additional features:
(i) additional light emitting module(s) directed to the base of the skull;
(ii) additional light emitting modules(s) to treat the eye area;
(iii) additional light emitting module(s) at the occipital region to treat the cervical spine;
(iv) EEG electrodes and associated monitoring means;
(v) shoulder supports;
(vi) additional securing means for securing the apparatus about an individual's head;
(vii) a transducer capable of measuring intracranial oxygenation;
(viii) alarm means for alerting an individual to a time to commence or cease treatment;
(ix) safety or cut-off means for cutting the power supply; and
(x) timing means to measure the length of a treatment period.

Preferably, the device of the present invention is portable.

Preferably, parts of the device that are in contact with the scalp are covered in a soft padding material to ensure it is comfortable to the end user. Extra soft material is placed over the areas in contact with the superior orbital ridge, the zygomatic arches, the peterous temporal bones, the occiput and the area over the superior sagittal sinus

According to a second aspect of the invention there is provided a method of making a customised transcranial phototherapy device according to the first aspect of the invention comprising the steps of:
(i) constructing a ring assembly the diameters of which are in accordance with measurements taken from the individual's cranium;
(ii) attaching light emitting light modules in a space defined between a first and second ring of the ring assembly at appropriate positions and in accordance with a condition to be treated.

It will be appreciated that the measurements of the individual's cranium could be obtained simply from using a tape measure or the like however, preferably, the method includes a first or pre-step of making a 3 dimensional impression of an individual's cranium.

Preferably, the step of making a 3 dimensional impression of an individual's cranium comprises making a direct impression of an individual's cranium by means of a plaster-cast or the like and then making a resin or wax or the like mould of the cast. The process is explained in greater detail in FIGS. 1 to 3 herein after. Alternatively, the step comprises making a 3D image by a CAD system and importing this into a CAD design.

It will be appreciated that the device of the invention can be said to be tailored or bespoke to each individual and accordingly will be not only a comfortable fit but will provide improved accuracy and efficiency of therapeutic light delivery.

Preferably, the model of the cranium is reviewed in conjunction with the individual's MRI or CT or SPECT scan or any other type of scan or diagnositic technique such as functional EEG that would locate and identify a pathological condition so as to ensure appropriate placement of the light emitting modules in the ring assembly.

According to a third aspect of the invention there is provided a method of treating, with the device of the first aspect of the invention, a neurological condition or state or pathology selected from the group comprising Picks disease, Alzheimer's dementia, Lewy body dementia, primary progressive aphasia, Huntington's chorea, Parkinson's disease, multiple sclerosis, multi-infarct dementia, brain injury due to trauma, brain injury due to hypoxia (both at birth and during anaesthesia/near drowning/industrial accident), cerebrovascular accident, central nervous pathology due to heavy metal poisoning, trisomy 21, viral encephalitis, viral meningitis, attention deficit hyperactivity disorder (ADHD), learning disability, autism and schizophrenia and depression.

Preferably the device of the first aspect of the invention may also be used to improve neurofeedback training, speed of analytical processing, memory, sexual function and general tiredness or malaise.

Preferably, the frequency, duration and therapeutic regimen is selected according to a user's requirements.

### BRIEF DESCRIPTION OF THE DRAWINGS

Embodiments of the invention are further described hereinafter with reference to the accompanying drawings, in which:

FIGS. 1 to 3 shows the steps of making an impression or mould of a human head, FIG. 1 shows an individual with a protective coating or cap covering the head and hair of the individual, FIG. 2 shows a cast formed to the contours of the individual and FIG. 3 shows the impression of FIG. 2 filled with a material to give a mould of the patient's cranium.

FIG. 4 shows modules placed around the impression of the patient's head.

FIG. 5 shows modules fixed to the bottom oval and to the upper oval of the mould.

FIG. 6 shows a finished customised product.

FIG. 7A shows a front view of one embodiment of a light emitting module and FIG. 7B shows a side view.

FIG. 8A shows a front view of an alternative embodiment of a light emitting module, FIG. 8B shows aside view of FIG. 8A, FIG. 8C shows a plan view of FIG. 8A and FIG. 8D shows a further view of FIG. 8A.

FIG. 9A shows a front view of a ring assembly and FIG. 9B shows a side angled view of FIG. 9A.

FIG. 10A shows a side view of a ring assembly and FIG. 10 B shows a plan view.

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

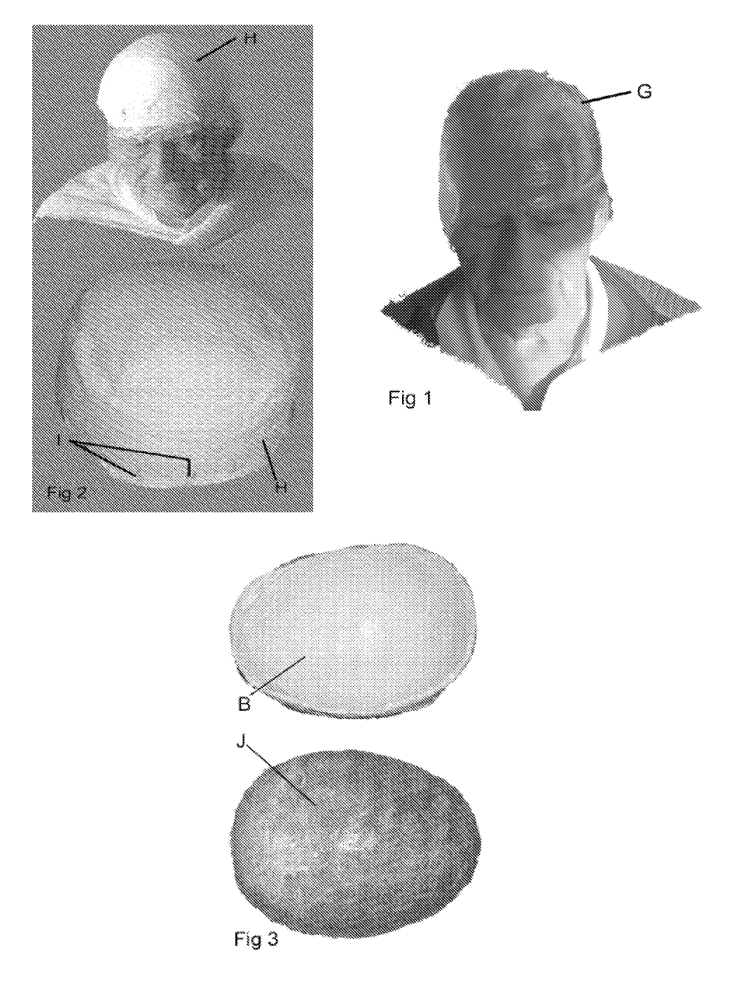

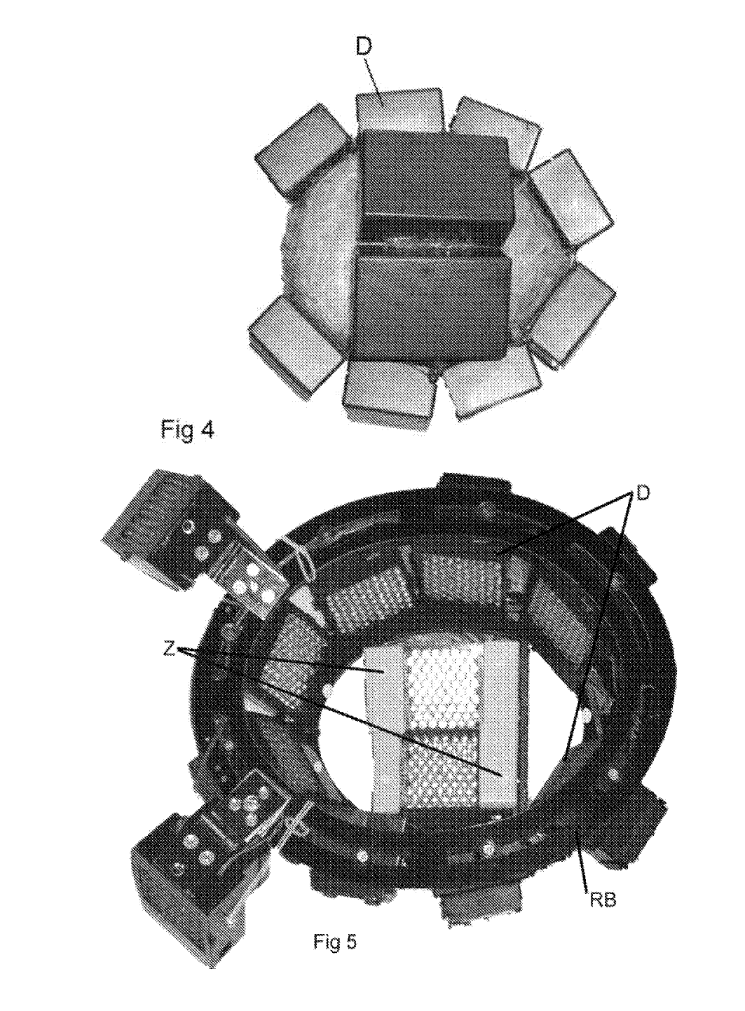

A goal of the present invention is to produce an individually moulded appliance which is custom made for each individual. FIGS. 1 to 3 shows the steps of making an impression or mould of a human head. Referring to FIG. 1, when taking an impression it is important to ensure the patient's head and hair is covered with a thin contour conforming cap G. An impression is then made of the individual's head using a suitable material that will harden in a few minutes to the contours of the individual's head to form a skull cal or hat H (FIG. 2). The impression can then be lifted clear of the patient's head having been clearly marked with the location of the ears, nose, eyes and occiput at regions I. The hardened impression B, which is substantially identical to the contour's of the individual's head is then filled with another suitable material such as wax or a resin or other material, ensuring the contours are adhered to, giving an exact mould of the patient's cranium J (FIG. 3). The mould of the head, J, is reviewed in conjunction with the functional MRI scan/SPECT scan. Light emitting modules D are held in place temporarily on the mould (FIG. 4). It is of note that the light emitting modules D may vary in shape, size and may be flexible to conform to the curvature of the head.

Modules D are then fixed to the bottom oval RB and to the upper oval RT so as to apply the modules D close to the scalp. The electrodes and cerebral oxygenation emitter and transducer Z are incorporated into the padding structure (FIG. 5). As the device is designed to follow the contours of the cranium good electrical contact will be made with each application.

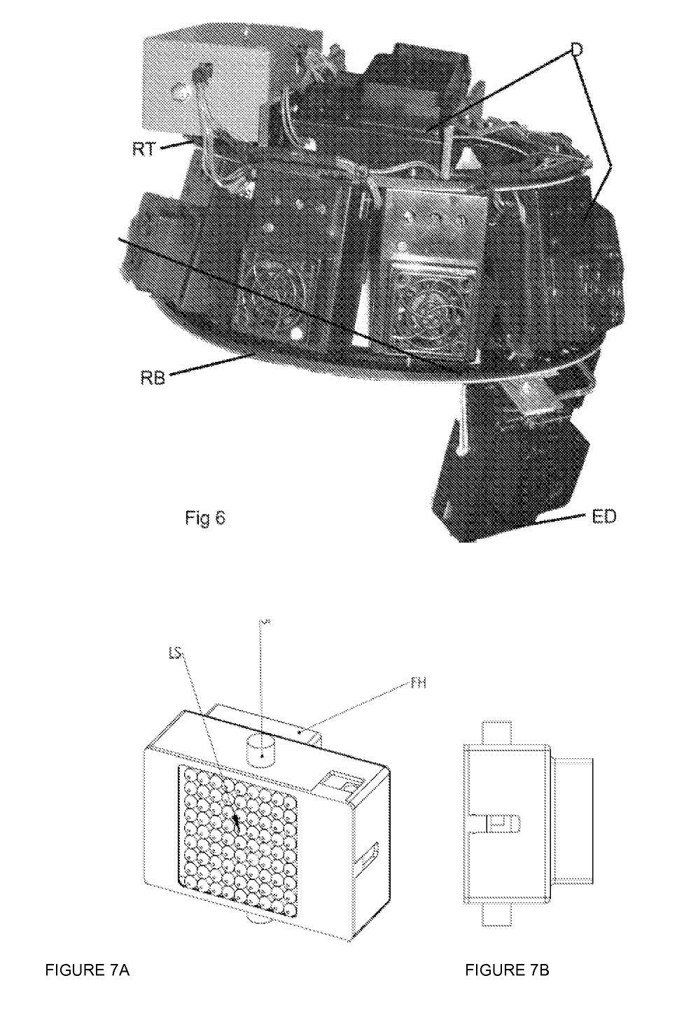

The finished customised product (FIG. 6) has the modules appropriately placed according the individual pathology and has a remote control unit controlling the unit. The apparatus may further optionally include eye modules ED which will treat co-existing ophthalmic pathology which is also age related.

A man skilled in the art will recognise that the physical steps depicted in FIGS. 1 to 6 can be equally discharged with the use of a 3 dimensional scanner and suitable CAD package.

Turning to FIG. 7A, there is shown an individual light module D, each module has a light source or a series of light sources which may vary in size and shape to ensure that optimal light administration to the skull occurs. The light source LS may be a rigid PCB based construction or may be flexible ensuring the close approximation of the light sources to the scalp. The fan housing FH facilitates the inclusion of a cooling fan, which cools the PCB ensuring the wavelength of the applied light is not shifted by the thermal heating effect of PN junctions. Spindle SP (FIG. 7B) is one of the options whereby a module may be connected to the top and bottom rings, TR and BR respectively via a connecting rod CR.

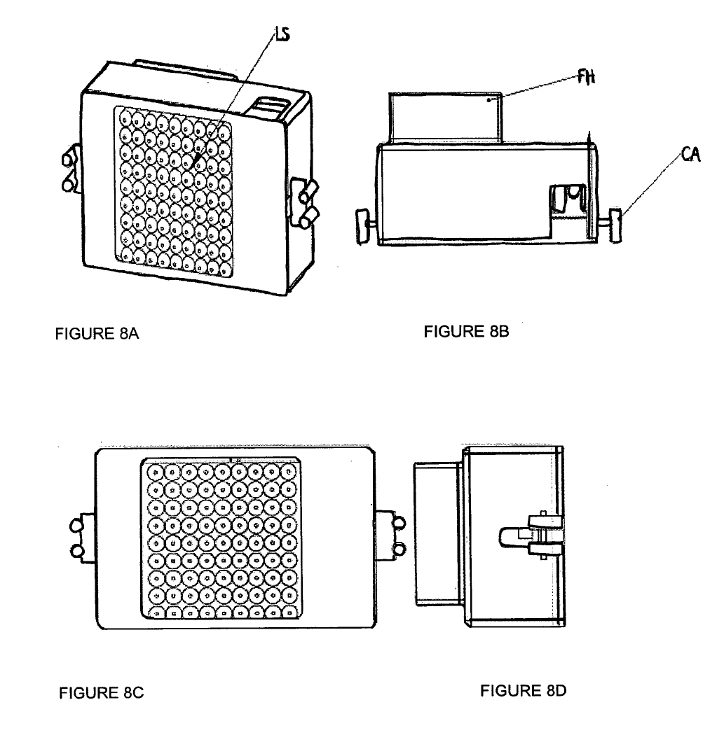

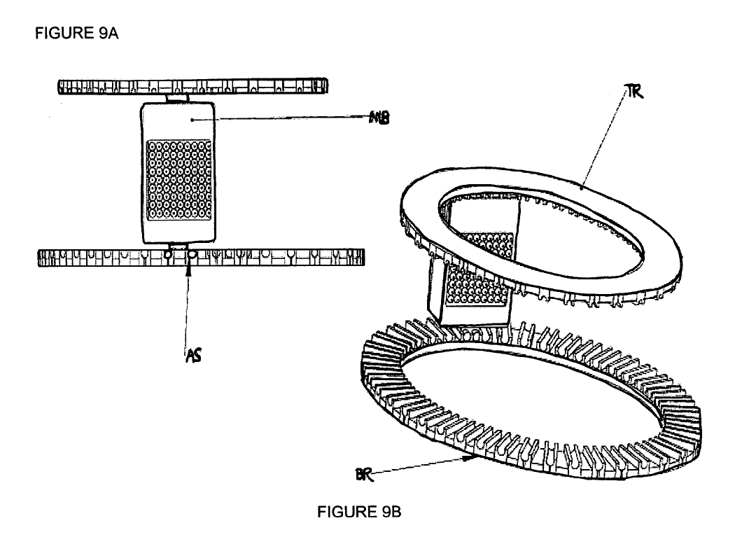

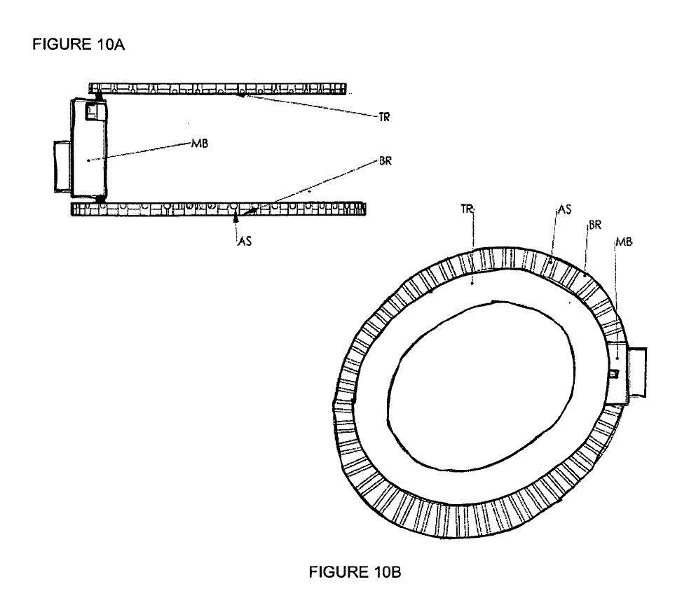

In an alternative embodiment of the light module D as seen in FIG. 8A, which has similar characteristics of the module of FIG. 7, the connecting means are different. The connecting means of the embodiment of FIG. 8 allow for an interlocking mechanism of the light module to upper and lower rings of a ring assembly and depends upon interlocking the circular portion CA of the module D, into the circular projections on the top and bottom rings, AS as seen on the ring assembly of FIGS. 9 and 10.

This mechanism of fixing the modules onto the ring assembly, whilst the rings are placed around a mould of the patient's head, will facilitate a rapid assembly of all components which when a small amount of resin is applied to the CA-AS joint will result in a rigid, customised durable device which can easily be used by an individual with cognitive decline or significant neurological tremor without the aid of a care giver.

Turning to FIGS. 9 and 10, for clarity only one of the modules is demonstrated, the flexible connection between CA and body of module MB will allow the module to tilt and follow the contours of the patient's head closely approximating the scalp. Rigidity of the construction is essential for reliability, as wiring which is repetitively bent is more likely to fail as seen in the prior art.

This construction facilitates easy placement of the applied light emitting modules accurately and effectively by a cognitively impaired or physically impaired individual without assistance, hence improving the compliance and effectiveness of the treatment with resultant improved therapeutic effect. In addition the customised applicator will ensure that the target of the phototherapy is the site of the pathological process.

Embedded within the customised transcranial phototherapy device (CTPD) are electrodes facilitating pre and post treatment EEG assessment, in addition infrared cerebral oxygenation transducers are intrinsic to the CTPD facilitating an optimal treatment protocol.

The ability to monitor the effect of the 1072 nm transcranial phototherapy by measuring both brain electrical activity and cerebral oxygenation allows the clinician to develop a personalised, individualised optimised treatment protocol which can be reviewed from time to time according to the progress of the patient. The monitoring facility also permits the detection of potential side effects due to the stimulated release of neurotransmitters by the 1072 nm light therapy.

The design of the custom made helmet will vary from individual to individual according to the clinical findings on the MRI, SPECT scan and functional EEG. Each individual, whilst having an identical clinical diagnosis will have subtle differences in their clinical presentation and findings which are representative of differences in anatomical sites of neuronal degeneration. Previous inventions have not addressed this issue, “one size does not fit all”.

The construction of the customised transcranial phototherapy device (CTPD) is with the use of resin impregnated fabric or resin alone. Multiple resins may be used, in a layered fashion. Alternatively silicone can be used to improve patient comfort. A person skilled in the art will recognise that any similar material can be used to achieve the identical result.

As stated herein before, the process involves placing a thin water resistant barrier between the patient and the resin impregnated fabric. The MRI/SPECT scans and functional EEGs are reviewed and the desired location for the modular 1072 nm light applicators chosen according to the desired clinical outcome. The modules are held in place and then the resin impregnated material is applied to the head.

Alternatively and preferred is the following process should be followed:
1. The patient has their functional MRI/SPECT scan or any other medical investigation which will identify the part of the brain which is malfunctioning.
2. The head and face is subject to a 3 dimensional scan, the hair being held close to the scalp with the use of a thin elasticised cap.
3. When scanning the head and face careful attention is paid to the anatomical land marks, nose, brow, palpabral apertures.
4. The medical scans are reviewed by an expert in the field, the placement of the light emitting modules reviewed accordingly.
5. Not only are the atrophied areas targeted but the area which communicates most with the atrophied area is targeted.
6. Using a CAD system, the 3D image of the head and face is imported into the CAD design.
7. The light emitting modules are put in place and then a customised upper and lower ring system is drawn according to the shape of the patient's head. Each ring system is unique to the individual patient.
8. The rings are then cut from a material which may be plastic, nylon, metal.
9. The modules are placed in the customised rings in accordance with the figures.
10. The parts of the customised that are in contact with the scalp are covered in a soft padding material to ensure the TCPA is comfortable to the end user.
11. Extra soft material is placed over the mould which represents the superior orbital ridge, the zygomatic arches, the peterous temporal bones, the occiput and the area over the superior sagittal sinus.
12. The light emitting modules and the soft support material are all held in place with a resinous/silicone material which becomes semi-rigid after hardening.
13. The ring, module assembly is slightly flexible which ensures comfort and durability.
14. Ancillary modules can be added for the treatment of the eyes and the medulla/brainstem according to clinical need.
15. An extension of the occipital region can be used to treat the cervical spine.
16. Shoulder support are optionally attached to the CTP device which allows the end user the option of reducing the load on the head. This will be beneficial to those individuals with cervical spine pathology.
17. The shoulder supports are spring loaded or repelling magnetic fields are used to reduce the weight carried by the neck without creating a gap between the applied light emitting modules and the scalp.

The result is a light weight, comfortable customised transdermal transcranial phototherapy device (CTPD) which is easily removed and placed on the patient's head. The moulded contours of the CTPD are such that device will always treat the desired area of the brain accurately and repetitively even when applied by a cognitively impaired individual. The similarity to that of a “hat” improves compliance and efficacy.

Preferably, the conditions that can be treated with the CTPD of the present invention are selected from the group comprising Picks disease, Alzheimer's dementia, Lewy body dementia, primary progressive aphasia, Huntington's chorea, Parkinson's disease, multiple sclerosis, multi-infarct dementia, brain injury due to trauma, brain injury due to hypoxia (both at birth and during anaesthesia/near drowning/industrial accident), cerebrovascular accident, central nervous pathology due to heavy metal poisoning, Trisomy 21, viral encephalitis, viral meningitis, Attention Deficit hyperactivity disorder (ADHD), learning disability, autism and schizophrenia and depression.

Individuals over the age of 40 years will also benefit from the CTPD, results have shown that they demonstrate an improvement in memory, speed of analytical processing, improved efficiency in the work place, greater productivity and improved sexual function and quality of erections in males, increased sex drive in females. Syndromes associated with chronic fatigue will also benefit from the CTPD, such as chronic fatigue syndrome, fibromyalgia and myeloencephalopathy (ME).

In addition the device is portable with an on-board battery supply which may also power an alarm. The alarm sounds and a verbal reminder in the preferred language of the user instructs the end user to use the CTP device at a predetermined time or times throughout the day. A visible flashing light external to the CTP may also be provided alerting the end user in the event they are hard of hearing.

The alarm sounds and the verbal/visual requirement to treat the end user occurs until the CTP device is placed on the head and treatment is carried out. The reminder message is instructive, telling the end user what to do with the CTP device.

The source of light used in the CTP device is preferably 1072 nm LEDs, lasers, laser diodes, light emitting polymers, light emitting organic or non-organic polymers and nanocrystals. Light emitting polymers and nanocrystals can be pumped with various wavelengths of light and the molecular structure of the polymer/nanocrystal is such that it only emits the desired peak wavelength in the region of 1072 nm. The range of wavelengths used will preferably be from 1020 nm to 1120 nm. Preferably, the light source can be pulsed or continuous wave.

Preferably, the pulse rate ranges from 250 Hz to 900 Hz.

Preferably, the average power output ranges from 2 mw/cm² to 1 W/cm².

Preferably, the treatment time ranges from 2 minutes to 30 minutes and preferably the treatment protocol ranges from 2-6 times daily treatment to twice weekly treatment. The duration of the treatment period may be according to a user's requirements.

### Example 1

Amyloid and neuroprotection, laboratory experiments were conducted and have demonstrated unequivocally the neuroprotective effect of 1072 nm light. Data has shown the ability of the 1072 nm light to inhibit amyloid formation which is well documented to be pathognomic of Alzheimer's Dementia.

4 patients who had benefited marginally from prior treatment with 1072 nm, were treated again with the customised transdermal applicator of the present invention, all demonstrated a marked improvement of the therapeutic effect of 1072 nm light. Results show that the physical design of the improved applicator is crucial to the improved efficacy. Most importantly compliance was improved significantly as the cognitively impaired individual was able to use the technology without the presence or input of a care giver.

### Example 2

Four patients with Picks disease were successfully treated with a mechanically adjustable 1072 nm helmet with a degree of success. At least one care provider was needed to apply the mechanically adjustable helmet which had adverse compliance effect in the event the carers were otherwise delayed. Not only was the compliance improved but there was an improvement of the cognitive function with the customised TPD, the MMSE score improved by 1-2 points over a period of 3 months in all cases.

### Example 3

Six patients with Alzheimer's dementia were treated with the improved customised device of the present invention, all of whom had not responded to the mechanically adjustable 1072 nm applicator. It was found that all the individuals were able to treat themselves and responded favourably to the concept. This improved compliance and hence efficacy of the delivery of the 1072 nm light.

### Example 4

Four patients with Parkinson's disease, who were unable to treat themselves with the 1072 nm mechanically adjustable helmet due to their tremor and who did not have regular skilled careers who could administer the adjustable headgear, were tested with the improved device of the present invention. They were able to use the CTPD unaided and hence the compliance and efficacy of the 1072 nm treatment was manifest within 30 days. In these individuals there was a marked improvement of their tremor and cognitive functioning.

### Example 5

Seven people who had suffered a cerebrovascular accident and presented with either paralysis or paresis of one side of their body were not able to apply the mechanically adjustable 1072 nm helmet without assistance, which reduced compliance and hence efficacy of the therapy. When given the CTPD they were able to treat themselves and an improvement of their daily living activity and verbal communication skills.

### Example 6

Two individuals with Huntington's Chorea, who were unable to use the mechanically adjustable 1072 nm helmet due to their involuntary head movements, were tested. Not only were they able to able to use the CTPD but they were able to use the device unaided which resulted in an improvement in their clinical state.

### Example 7

Two patients with the genetic abnormality trisomy 21, 2 individuals were treated with the CTPD to good effect, improvement in intellectual ability was noted after a month. Their level of autonomy improved from before treatment. Three patients with Attention Deficit hyperactivity disorder (ADHD), responded favourably after 6 weeks of twice weekly to 5 times weekly treatments. Daily or even twice daily treatments are anticipated to be as effective. In six individuals with learning disability and autism, who used the CTPD were found to have improved concentration, improved behaviour issues and improved intellectual performance. Two individuals suffering from schizophrenia, who had incomplete response to antipsychotic medication were treated with the 1072 nm technology and CTPD with good effect, there was an improvement of their psychotic features after a wee of daily treatments. The improvement was also noted in individuals who were hallucinating as part of their neurodegenerative process. Eight patients suffering from depression, who had incomplete response to antidepressant medication were treated with the CTPD with good response after 4 weeks of daily treatment. Two of the patients had treatment resistant depression due to substance misuse (eg. Amphetamines, cocaine, heroine etc).

### Example 8

The inclusion of the EEG electrodes and the cerebral oxygenation apparatus facilitated the individualisation of the treatment protocol for suffers of multiple sclerosis, as well as allowing neurofeedback training to be included in the therapeutic management portfolio. These combination of therapeutic intervention and neurofeedback potentates the effect of the 1072 nm treatment especially once the neuronal regeneration had become established.

Treatment of normal individuals with intellectually demanding occupations used the CTPD over a period of 8 weeks. They all reported improvement in their efficiency, increase in libido and quality of erections (in men). They all reported an improvement in vigour and energy and a decrease in anxiety. Four individuals suffering from fibromyalgia/ME were treated daily, an improvement their feeling of wellbeing occurred within the first few days, improvement in energy and mood occurred within the first month.

1. A phototherapy device comprising a first substantially circular ring and a second substantially circular ring that is of a smaller diameter than the first ring, each ring being sized and shaped so as to be approximately commensurate with the circumference of an individual's head and in use is able to fit over the individual's head, each ring being provided with a plurality of engaging means for receiving and securing a plurality of light emitting modules therebetween, wherein the light emitted by the light emitting modules is centred around a peak wavelength of 1072 nm.

2. A phototherapy device according to claim 1 wherein the first and second rings are constructed of a rigid or semi-rigid flexible material selected from the group consisting of: silicone, resin, plastics and rubber.

3. A phototherapy device according to claim 1 wherein the first and second rings each have an outer surface that is most remote from the plurality of light emitting modules and an inner surface in proximity to the plurality of light emitting modules and wherein the outer surfaces of the first and second rings are substantially flat and wherein the inner surfaces of the first and second rings are profiled according to the type of engagement means employed in the device that secure the light emitting modules in a space defined between the two rings.

4. A phototherapy device according to claim 1 wherein the engaging means of the first and second rings that engage with the plurality of light emitting modules are in the form of complementary interlocking male and female projections and receiving units.

5. A phototherapy device according to claim 4 wherein the first and second rings each have an inner surface in proximity to the plurality of light emitting modules and wherein the light emitting modules are provided with at least one or more cylindrical projections that are received into complementarily shaped receiving sections or grooves or slots provided on the inner surface of the first and second rings.

6. A phototherapy device according to claim 4 wherein the first and second rings are provided with a plurality of cylindrical projections that are received into complementarily shaped receiving sections or grooves or slots provided on the light emitting modules.

7. A phototherapy device according to claim 1 wherein the engaging means of the first and second rings that engage with the plurality of light emitting modules are in the form of a rod and spindle arrangement, the plurality of light emitting modules being provided with a hollow projection on a top and bottom surface of its casing into which a rod maybe inserted, the first and second rings being provided with means for securing the rod.

8. A phototherapy device according to claim 1 wherein the plurality of light emitting modules are releasably engaged with the first and second rings so that they may be replaced.

9. A phototherapy device according to claim 1 wherein the plurality of light emitting modules comprises a housing or casing within which there is provided a plurality of light emitting means selected from the group consisting of: LEDs, lasers, laser diodes, light emitting polymers, light emitting organic or non-organic polymers and nanocrystals.

10. A phototherapy device according to claim 9 wherein the light emitting means are LEDs.

11-12. (canceled)

13. A phototherapy device according to claim 1 wherein the light is pulsed or is a continuous wave.

14. A phototherapy device according to claim 1 wherein the light emitting module further includes a fan or cooling element.

15. A phototherapy device according to claim 1 further including one or more of the following:
(i) at least one light emitting module directed to the base of the skull;
(ii) at least one light emitting modules to treat the eye area;
(iii) at least one light emitting module at the occipital region to treat the cervical spine;
(iv) EEG electrodes and associated monitoring means;
(v) shoulder supports;
(vi) additional securing means for securing the apparatus about an individual's head;
(vii) a transducer capable of measuring intracranial oxygenation;
(viii) alarm means for alerting an individual to a time to commence or cease treatment;
(ix) safety or cut-off means for cutting the power supply; and
(x) timing means to measure the length of a treatment period.

16. A phototherapy device according to claim 1 that is portable.

17. (canceled)

18. A method of making a phototherapy device according to claim 1 comprising the steps of:
(i) constructing a ring assembly comprising at least two rings, wherein the at least two rings each have a diameter which is in accordance with measurements taken from the individual's cranium;
(ii) attaching emitting light modules in a space defined between a first and second ring of the ring assembly at appropriate positions and in accordance with a condition to be treated.

19. A method according to claim 18 further including a step of making a 3 dimensional impression of an individual's cranium before step (i).

20. A method according to claim 19 wherein the step of making a 3 dimensional impression of an individual's cranium comprises making a direct impression of an individual's cranium by means of a plaster-cast and then making a resin or wax mould of the cast or comprises making a 3D image by a CAD system and importing the 3D image into a CAD design.

21. (canceled)

22. The method according to claim 18 wherein a model of the cranium is reviewed in conjunction with a diagnostic technique that would locate and identify a pathological condition of the individual so as to ensure appropriate placement of the light emitting modules in the ring assembly.

23. A method selected from:
a) methods of treating a neurological condition or state or pathology selected from the group consisting of: Picks disease, Alzheimer's dementia, Lewy body dementia, primary progressive aphasia, Huntington's chorea, Parkinson's disease, multiple sclerosis, multi-infarct dementia, brain injury due to trauma, brain injury due to hypoxia, cerebrovascular accident, central nervous pathology due to heavy metal poisoning, trisomy 21, viral encephalitis, viral meningitis, attention deficit hyperactivity disorder (ADHD), learning disability, autism and schizophrenia and depression, the method comprising shining light emitted from the device of claim 1 on to an appropriate region of an individual's cranium; and
b) methods of improving a condition selected from the group consisting of: neurofeedback training, speed of analytical processing, memory, sexual function and general tiredness or malaise, the method comprising shining light emitted from the device of claim 1 on to an appropriate region of an individual's cranium.

24-25. (canceled)

26. A phototherapy device comprising a first substantially circular ring and a second substantially circular ring that is of a smaller diameter than the first ring, each ring being sized and shaped so as to be approximately commensurate with the circumference of an individual's head and in use is able to fit over the individual's head, each ring being provided with a plurality of engaging means for receiving and securing a plurality of light emitting modules therebetween; wherein the first and second rings each have an outer surface that is most remote from the plurality of light emitting modules and an inner surface in proximity to the plurality of light emitting modules and wherein the outer surfaces of the first and second rings are substantially flat and wherein the inner surfaces of the first and second rings are profiled according to the type of engagement means employed in the device that secure the light emitting modules in a space defined between the two rings.
